(19) United States
(12) Patent Application Publication
(10) Pub. No.: US 2014/0322724 A1
(43) Pub. Date: Oct. 30, 2014

(54) HOMOGENEOUS COMPETITIVE LATERAL FLOW ASSAY

(71) Applicant: Kieran WALSHE, County Meath (IE)

(72) Inventor: Kieran Gerard Walshe, Ratoath (IE)

(21) Appl. No.: 14/364,698

(22) PCT Filed: Dec. 13, 2012
(86) PCT No.: PCT/IE2012/000053

§ 371 Date: Jun. 12, 2014

(30) Foreign Application Priority Data
Dec. 13, 2011 (IE) S2011/0547

(51) Int. Cl. G01N 33/543

(52) U.S. Cl. CPC G01N 33/54393
USPC 435/7.1; 436/501; 422/69; 435/287.2

(57) ABSTRACT

A patient or animal side method and assay for eliminating the hook effect in the detection of a target analyte such as an acute phase protein in a bodily fluid in which the target analyte comprises a member of a specific binding pair comprising applying the sample to a solid phase carrier material, generating a signal in accordance with downstream movement of the labelled first or second members and the target analyte to bind with the complimentary immobilised first or second members, and detecting the presence of the target analyte in accordance with the signal generated at the complimentary immobilised first or second members.

### INTRODUCTION

This invention relates to an assay and, in particular, to a lateral flow assay and a device for performing the lateral flow assay.

### BACKGROUND OF THE INVENTION

Immunoassays generally employ one or more select antibodies to detect analytes or antigens of interest. The high specificity and affinity of antibodies for a specific antigen allows the detection of analytes by a variety of immunoassay methods.

In general immunoassays rely on specific binding pair members where each specific binding pair member is one of two different molecules (sbp members) having an area which specifically binds to and is complimentary with a portion of the other molecule. The two molecules are related in such a way that their binding to each other enables them to distinguish their binding partner from other assay constituents. Complimentary sbp members bind to each other such as antigen (analyte) and antibody against the analyte and ligands and receptors (e.g. biotin and avidin/strepavidin).

Lateral flow (immuno)assays (LFA or LFIA), hereinafter referred to as lateral flow assays, are important diagnostic tools and are widely used for the detection of a wide range of analytes. Typically, lateral flow assays are prefabricated strips of a solid phase carrier material containing dry reagents that are activated by applying a fluid sample. Lateral flow assays can be used for the diagnosis of conditions in human and veterinary medicine e.g. pregnancy detection, failure of internal organs (e.g. heart attack, renal failure or diabetes), infection and contamination with specific pathogens including bio warfare agents.

Immunoassays can be prone to interferences that compromise the specificity and sensitivity of the immunoassay. For example, the hook effect, or high dose hook effect, describes a wrong low measurement or false negative assay result for analytes which are present in a specimen in a very high concentration—if the analyte concentration is too high, antibody binding sites can become fully occupied or saturated and additional analyte molecules cannot be measured within the limit of the binding curve leading to false negatives or falsely low quantitative measurements depending on whether the test is qualitative or quantitative.

The hook effect is a common occurrence in many immunoassays but is a particular problem in those assays that are homogeneous i.e. where the target analyte and the detection antibody are present at the same time and no separation step is required. The outcome is that the target analyte present at a high concentration is, at best, seriously underestimated and, at worst, a false negative result for the target analyte is obtained thereby falsely indicating that the patient has normal levels of the target analyte. Lateral flow assays and many other homogenous assays are particularly prone to interference from the hook effect.

In order to avoid the hook effect many assays are designed using complex multi-step or heterogeneous assay formats which employ wash or sample dilution steps to eliminate target analyte excess. However, multi-step assay formats are, in general, unsuitable for use outside a laboratory environment where laboratory personnel can perform the complex steps.

More generally, in known assays, many bodily fluids to be analysed (e.g. whole blood), must be subjected to sample processing steps prior to analysis in order to prevent other components in the sample from compromising the efficacy of the assay. Accordingly, as experienced skilled laboratory staff and automated equipment is required to perform the sample processing, the immunoassays cannot be performed patient side in human medicine or animal side in veterinary medicine. In particular, due to the need to extract serum or plasma from whole blood for analysis, assays cannot be performed patient or animal side on untreated whole blood samples.

Lateral flow assays are in general employed for qualitative analyses only as, apart from the complex steps required to eliminate the hook effect and other interferences outlined above, quantitative analyses in general require additional complex instrumentation and laboratory techniques—and skilled technicians to perform the quantitative analysis.

Some quantitative lateral flow assays are available for a limited range of analytes but require the use of difficult to operate optical reader devices into which the assay must be inserted in order to obtain a quantitative readout. However, a disadvantage with such systems is the need for the expensive and complex reader devices resulting in increased assay costs. Moreover, quantitative optical devices or quantitative assays requiring the user to interpret a colour change cannot be used with whole blood due to its colour and semi-opacity.

In short, various human and animal analytes used for diagnostic purposes are subject the hook effect and other interferences rendering them unsuitable for patient side or animal side qualitative, quantitative or semi-quantitative analyses with one-step non-instrument based assays such as lateral flow assays.

Examples of human analytes subject to the hook effect include C-reactive protein (CRP), alpha fetoprotein (AFP), cancer antigen 125 (CA 125), prostate specific antigen (PSA), ferritin, prolactin, myoglobin and thyroid stimulating hormone (TSH) and the human pregnancy hormone HCG.

An example of such an analyte common to human and animal medicine is acute phase proteins (APP's) in which blood tests are performed to identify elevated concentrations of APP's to diagnose infection, inflammation, trauma, burns, malignancies and general tissue damage. Examples of APP's known for diagnostic and prognostic purposes include haptoglobin, CRP and serum amyloid A (SAA). More particularly, analysis of APP levels has been shown to have utility for diagnostic purposes in, inter-alia, cattle, pigs, cats, dogs, chickens, horses and humans.

For example, equine SAA is present at trace levels in healthy horses but increases rapidly following tissue injury, infection, trauma and arthritis. Moreover, determination of declining SAA levels in horses may be a useful prognostic tool to assess reconvalescence of horses recovering from infections such as respiratory infections or during recovery after injury.

Similarly, elevated levels of SAA in humans can be indicative of inflammation and an underlying infection. However, in order to eliminate the interferences outlined above, known assays for human APP's must employ laboratory based assay methods such as radio-immunoassays (RIA), nephelometry and turbidimetry rendering the assays slow and expensive—and prohibitive where large populations must be tested.

Accordingly, due to the speed with which APP levels can rise and fall in animals and humans, the unsuitability of known assays for performing rapid and reliable qualitative and quantitative assays in-situ (e.g. SAA assays animal or patient side) prevents the use of such potentially useful diagnostic tools.

Due to their simplicity, LFA's are preferred for animal and patient side assays. Where analytes are of low molecular weight and have one epitope only, i.e. a hapten, competitive LFA's are employed by those skilled in the art—particularly where the hapten is present in the pg-ng/ml range. The response is inversely proportional to the amount of analyte in the sample. Conversely, for analytes with more than one epitope, sandwich assays are employed by those skilled in the art so that the response is directly proportional to the amount of analyte in the sample. However, due to the hook effect exhibited by many analytes such as APP's at low to moderate μg/ml levels and most analytes at high μg/ml levels and upwards, the sandwich assay is generally regarded as only being useful for detection of analytes that are present in quantities less than μg/ml levels.

In short, for the reasons outlined above, LFA's have not been employed and have been neglected by those skilled in the art for quantitative and/or semi-quantitative detection of analytes such as APP's in human and veterinary medicine for diagnostic purposes.

### SUMMARY OF THE INVENTION

According to the invention there is provided a method for the detection of a target analyte in a sample in which the target analyte comprises a member of a specific binding pair comprising:
applying the sample to a solid phase carrier material, the solid phase carrier material having labelled first or second members of the specific binding pair thereon and complimentary immobilised first or second members of the specific binding pair downstream of the labelled first or second members of the specific binding pair,
generating a signal at the complimentary immobilised first or second members of the specific binding pair in accordance with downstream movement of the labelled first or second members to bind with the complimentary immobilised first or second members, and
detecting the presence of the target analyte in accordance with the signal generated at the complimentary immobilised first or second members.

Suitably, the analyte comprises an immunologically detectable analyte.

Preferably, the analyte is a human analyte. Alternatively, the analyte is an animal analyte. The animal analyte can be sampled from the group comprising horses, cows, dogs, cats, pigs, cattle, goats, sheep, donkeys, and llamas.

Preferably, the analyte comprises a protein. More preferably, the protein comprises an acute phase protein. Most preferably, the acute phase protein comprises serum amyloid A. Optionally, the analyte comprises a hormone.

Suitably, the sample comprises a liquid sample and the method further comprises the step of pre-filtering the liquid sample.

Preferably, the liquid sample is a bodily fluid and more preferably the bodily fluid is selected from the group comprising blood, plasma, serum, milk, colostrums, peritoneal fluid, synovial fluid and urine. Most preferably, the bodily fluid comprises whole blood.

Advantageously, the signal is generated at at least one test line and preferably signals are generated at a plurality of test lines.

Suitably, the analyte is quantitatively detected in accordance with the signal generated at the test line. Alternatively, the analyte is semi-quantitatively detected in accordance with the signal generated at the test line.

Suitably, the immunologically detectable analyte comprises the first member of the specific binding pair and the second member of the specific binding pair comprises labelled antibody. Preferably, the labelled antibody comprises monoclonal antibodies. Optionally, the antibody comprises polyclonal antibodies.

In an alternative embodiment of the invention, the specific binding pair comprises antibody fragments such as FAB or FAB₂, receptors, complementary nucleic acid sequences, aptamers and the like.

Preferably, the labels comprise visual labels. More preferably, labels are selected from the group comprising gold, latex, silver, liposomes, selenium, carbon and dyes.

Alternatively, the labels are selected from the group comprising non-visual fluorescent or biochemiluminescent labels, quantum dots or upconverting phosphor technology particles.

In a preferred embodiment, the invention further comprises the step of diagnosing a condition in a human or animal in accordance with the signal generated. Preferably, the condition comprises inflammation or infection.

The invention also extends to a method further comprising the step of reading the signal generated with a reader device. Preferably, the reader device comprises a handheld reader device. More preferably, the handheld reader device comprises a mobile phone.

The invention also extends to a lateral flow assay device for eliminating the hook effect in the detection of a target analyte in a sample in which the target analyte comprises a member of a specific binding pair comprising:
a solid phase carrier material;
labelled first or second members of the specific binding pair on the solid phase carrier material;
complimentary immobilised first or second members of the specific binding pair on the solid phase material downstream of the labelled first or second members of the specific binding pair;
a signal being generatable at the complimentary immobilised first or second members of the specific binding pair in accordance with downstream movement of the labelled first or second members to bind with the complimentary immobilised first or second members, and
a pre-filter on the solid phase carrier material to remove interferences from the sample.

Preferably, the labelled first or second members of the specific binding pair on the solid phase carrier material comprises a labelled target analyte antigen and the complimentary immobilised first or second members of the specific binding pair on the solid phase material downstream of the labelled first or second members of the specific binding pair comprises an antibody to the antigen.

Optionally, the antigen comprises a protein. Preferably, the protein comprises an acute phase protein. More preferably, the acute phase protein comprises a human acute phase protein.

Alternatively, the acute phase protein comprises an animal acute phase protein.

Suitably, the animal acute phase protein is selected from the group comprising equine, bovine, canine, feline, porcine, goat, sheep, donkey and llama acute phase protein.

Preferably, the acute phase protein comprises serum amyloid A.

In a further embodiment, the invention also extends to the use of a lateral flow assay device as hereinbefore defined in human or animal diagnostics.

Suitably, the lateral flow device is employed in the diagnosis of inflammation or infection in humans or animals and preferably is employed in the animal or patient side diagnosis of inflammation or infection in humans or animals.

The invention also extends to a method for eliminating the hook effect in the detection of a target analyte in a sample as hereinbefore defined in which the sample is applied directly to the solid phase carrier material without pre-treatment and the method comprises the step of prefiltering the sample to remove blood cells at the solid phase carrier.

The assay of the invention overcomes the hook effect encountered with analytes such as APP's that are present in biological fluids such as blood. The simple and rapid assay can be used for the quantitative and/or semi-quantitative analysis of such analytes animal or patient side in a cost-effective and easy manner without requiring sophisticated laboratory techniques. Moreover, as it is the concentration of analyte that causes a hook effect, the multi-purpose assay of the invention can also be used for the quantitative and semi-quantitative detection of other molecules including lower molecular weight molecules/haptens such as toxins and hormones that occur at lower levels than SAA or other APP's if desired.

The assay of the invention enables detection of a molecule regardless of whether the molecule occurs at pg/ml levels or at mg/ml levels. Although the assay of the invention is suitable for use animal and patient side with biological fluids such as whole blood, the assay can be used animal and patient side or in a laboratory with other biological fluids including serum and plasma.

However, importantly and surprisingly the invention enables detection of target analytes using whole blood, with direct application of the blood sample to a test device without the need for any prior sample processing such as dilution or washing. Accordingly, assays can be performed in-situ in the presence of a patient or animal side in veterinary applications.

Therefore, the LFA of the invention can be used for the detection of analytes of varying molecular weight and the quantification and/or semi-quantification of the analytes over a range of concentrations from pg/ml up to μg/ml and mg/ml levels whilst overcoming the high dose hook effects encountered using known sandwich assay formats. Accordingly, the assay of the invention can be used for rapid animal and patient side diagnostic purposes.

The multi-purpose assay of the invention can be used qualitatively, semi-quantitatively and quantitatively to detect the presence of analyte antigens in human and veterinary purposes. As the assay of the invention is a homogenous competitive assay, the assay is cost-effective, rapid and easy to use by professionals and non-professionals alike in animal and patient side situations. Immediate results can be obtained without requiring laboratory processing of samples. Where the assay of the invention is used for the detection of APP's such as SAA in fluid samples, immediate diagnoses can be made to enable immediate treatment of animals and humans alike.

Moreover, as the assay of the invention is adapted to overcome interferences such as the hook effect usually encountered with untreated fluid examples such as whole blood, serum or plasma the complex pre-treatment of the fluid samples is not required thereby further enhancing the immediacy of the results achievable with the assay of the invention.

The assay of the invention eliminates false negative results encountered with assays of the prior art due to the hook effect. Semi-quantitative or quantitative results can be achieved without the need of trained laboratory personnel and sophisticated equipment. Complex reader devices are not required while the assay is suitable for diagnostic and prognostic purposes. Nevertheless, the method and assay of the invention can be used with reader devices if desired including mobile reading technology devices such as handheld devices and mobile phone devices.

Due to the simplicity and cost-effectiveness of the assay of the invention, the assay can be used for diagnostic purposes in large human and animal populations.

### BRIEF DESCRIPTION OF THE DRAWINGS

The invention will now be described, by way of example only, with reference to the accompanying drawings and Examples in which:

FIG. 1 is a diagram illustrative of the hook effect in which analyte concentration is plotted on the X-axis and analyte signal is plotted on the Y-axis with the signal decreasing at high concentrations;

FIG. 2 is a schematic representation of test results for SAA obtainable using the assay of the invention namely an invalid result, a normal result (four Test Lines visible (including Control Line)—normal analyte levels), a mild inflammation result (three Test Lines (including Control Line) visible—mildly raised analyte levels), a moderate inflammation result (two Test Lines visible (including Control Line)—moderately raised analyte levels) and a severe inflammation result (one Test Line visible (Control Line only)—severely raised analyte levels), and

FIG. 3 is a plan view from above of an SAA test strip suitable for use in performing the assay.

### DETAILED DESCRIPTION OF THE INVENTION

The present invention enables detection of any analyte and particularly immunologically detectable analytes regardless of concentration range but especially analytes that occur at levels that would normally cause a hook effect. Moreover, analyses can be performed on whole untreated bodily fluids such as, inter alia, blood, colostrums, milk, peritoneal fluid, synovial fluid and urine.

In the following description and Examples, the invention is described with reference to human and veterinary diagnostics and, in particular, with reference to human, equine and feline SAA. However, as will be appreciated by those skilled in the art, the LFA and devices of the invention are suitable for use with a wide range of animals including but not limited to pigs, cattle, goats, sheep, donkeys, llamas and other domestic animals such as cats and dogs.

In addition, as indicated above, although the following Examples describe the analysis of SAA for diagnostic purposes, the LFA of the invention is suitable for use with a wide range of analytes such as CRP and other blood markers such as hormones, including progesterone, pregnant mare's serum gonadotropin (PMSG), oestrone sulphate and immunoglobulin G (IgG), an essential component in colostrum for new born foals, and cortisol.

The LFA of the invention in the following Examples employs a competitive format in which antibodies to the analyte are typically used for recognition. However, as will be appreciated by those skilled in the art, other binding partners can be used including but not limited to receptors, complementary nucleic acid sequences, aptamers and the like.

Various competitive formats can be employed in the LFA devices of the invention. In a one step competitive format, antibody is sprayed at the test line(s), a mixture of sample analyte and labelled analyte or an analogue of the analyte react at the conjugate pad and the sample analyte and labelled analyte compete for binding sites on the antibody at the test line(s). Alternatively, an analyte or analogue of the target analyte can be applied/sprayed at the test line(s) and a mixture of labelled antibody and sample analyte can react at the conjugate pad prior to migrating along the test strip to the test lines. In this format, by reacting sample with antibody labelled gold, sensitivity is increased thereby giving the sample analyte a “head start” for binding to the antibody.

Moreover, the assay of the invention is exemplified with reference to cassette type device. However, other forms of the assay can also be used such as test strips for dipping into body fluids.

Where reference is made to a monoclonal antibody, polyclonal antibodies or antibody fragments could similarly be used.

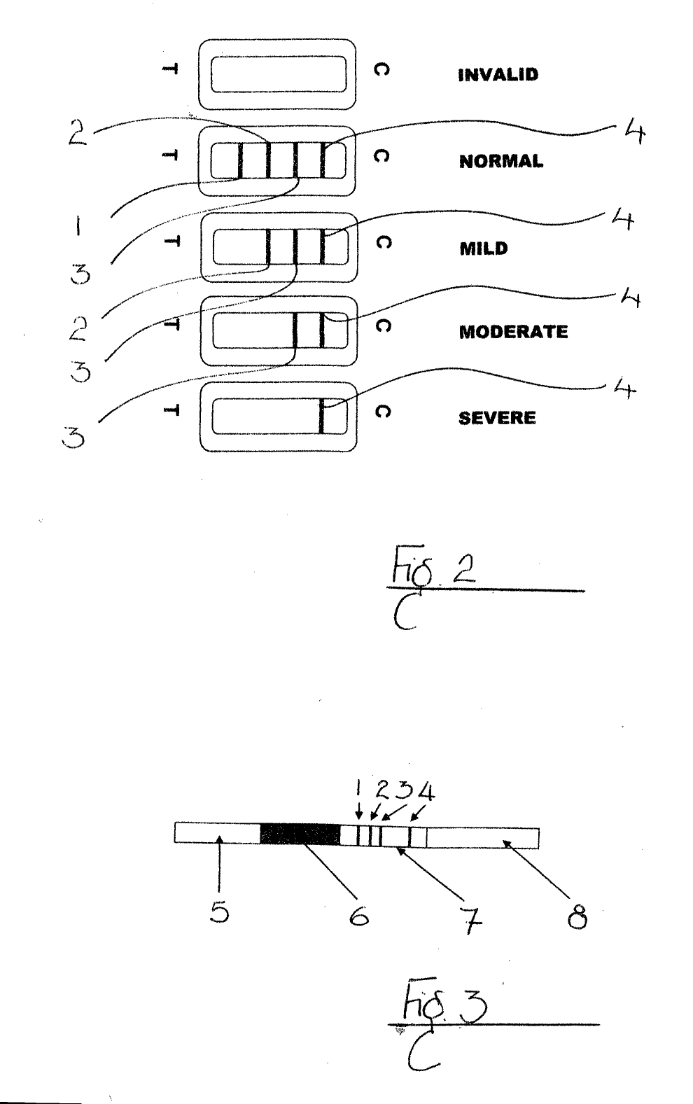

The assay device described uses many components which will be familiar to those skilled in the art. In the Examples outlined below (and as shown in FIG. 2), three Test Lines 1,2,3 respectively were printed with purified SAA. The concentration of SAA used for deposition on the Test Lines 1,2,3 can either be the same or can be graduated so that the first Test Line (Test Line 1) has a lower SAA concentration than the second Test Line (Test Line 2) which in turn has lower concentration of SAA than the third Test Line (Test Line 3) although other combinations are possible. Alternatively, the sequence of SAA concentration can be reversed so that the SAA concentration of Test Line 1 can be higher than the concentration of SAA at Test Line 2 which in turn is higher than the concentration at Test Line 3 etc.

In addition, an optional Control Line 4 can be added which acts as a procedural control. This Control Line 4 can use gold labels which will give a coloured signal identical to the gold used for Test Line signal generation. In an alternative embodiment, the Control Line 4 uses a different coloured particle such as blue latex of silver particles which can give a yellow or orange colour. The Control line 4 is printed with an antibody that reacts with a protein coated on to the particle selected for use as a control signal generator.

As indicated above, for the purpose of the Examples outlined below, three test lines 1,2,3 were employed. However, any number of lines can be employed in the LFA of the invention e.g. 1-5 or more lines are feasible. However, at least two lines are preferred for visual quantification.

The concentration of SAA used for deposition on the Test Lines 1,2,3 can either be the same or can be graduated so that Test Line 1 has lower SAA then Test Line 2 which in turn has lower SAA than Test Line 3. Alternatively, the sequence of SAA concentration can be reversed as outlined previously.

Accordingly, the antibody-labelled gold migrates along the test strip until it reaches the Test Line 1 where the antibody-labelled gold reacts with the SAA printed on Test Line 1 giving a clear red signal on this line. Unreacted antibody labelled gold then migrates to Test Line 2 giving a second clear distinct coloured line. Further unreacted antibody-labelled gold then migrates past Test Line 2 and reacts with Test Line 3 on the test strip giving a third coloured line.

If desired, the LFA device of the invention can be adapted so that the intensity of the colour generated at Test Lines 1,2,3 can be either of equal intensity or can be graduated so the colour on Test Line 1 is weaker than on Test Line 2, which in turn is weaker than on Test Line 3 with the reverse scenario being possible if desired.

If analyte is present in the sample, the target SAA analyte reacts with the antibody on the antibody-labelled gold in proportion to the amount of SAA present until such time as all the antibody binding sites on the gold are occupied by SAA in the sample. However, as SAA increases to levels above a selected threshold, the amount of the antibody-labelled gold available for reaction with the SAA on the test strip is reduced because analyte binding sites on the antibody-gold conjugate previously available for reaction with SAA on the test line are now occupied by SAA present in a sample.

Accordingly, at defined SAA blood levels, SAA bound to the antibody-labelled gold prevents the antibody-labelled gold from reacting with the SAA on Test Line 1 so that no colour appears on Test Line 1, so that first line effectively “disappears” or is no longer visible. As the SAA concentration in the sample increases further, fewer free analyte binding sites exist on the antibody-gold particles so that at certain levels of SAA both Test Line 1 and Test Line 2 “disappear” or is no longer visible so that the red colour is seen at Test Line 3 only. As SAA levels in the sample increase even further then Test Line 3 also “disappears” giving no red lines at all with the control line 4 only remaining visible.

As outlined further below, in the case of SAA determination, three lines can be used to determine if an animal, for example a horse, is normal, i.e. little or no inflammation as indicated by three visible test lines, has mild inflammation as indicated by two visible test lines, has moderate inflammation as indicated by one visible test line and has severe inflammation as indicated by no visible test lines 1,2,3.

The assay can therefore be configured so that each Test Line 1,2,3 can be used to represent specific concentration ranges e.g. at SAA levels of less than 10 μg of SAA/ml of blood, Test Lines 1,2,3 are clearly visible, at SAA levels from 10-50 μg/ml Test Lines 2,3 are visible, while at SAA levels from 50-200 μg/ml only Test Line 3 is visible and at SAA levels greater than 200 μg/ml no test lines are visible. As will be appreciated by those skilled in the art, these ranges can be calibrated to increase or decrease the ranges as required.

By way of example, in the detection of inflammation using SAA levels, one drop of blood, serum or plasma can be added to the end of a test strip followed by two drops of a buffer to act as a “chaser” to help move the sample along the test strip. The actual volume of sample applied can vary from as a little as about 1 μl up to about 100 μl.

The LFA and devices of the invention can be adapted to work with a specific sample volume. The assay of the invention can easily be adapted and optimised according to the volume of blood applied so that the assay of the invention can perform to the required specification.

Alternatively, the sample to be analysed can be pre-diluted in the “chaser” and the whole sample added slowly to the end of the test strip. A further option is to pre-dilute the sample and dip the test strip into the diluted sample.

Structurally, a typical LFA format suitable for use in the LFA devices of the invention is made up of a surface membrane layer to carry the sample from a sample application pad via a conjugate release pad along a strip encountering a detection zone to an absorbent pad. The membrane is attached to a plastic or nylon basic layer to allow cutting and handling to provide added robustness. In addition, robustness can also achieved by housing the strips in a plastic holder where only the sample application window and a reading window are exposed although test strips are used without need for this plastic housing. The membrane strips can be produced from nitrocellulose, nylon, polyethersulfone, polyethylene or fused silica although other materials known to those skilled in the art are possible.

At one end of the membrane strip a sample application pad is provided. The sample application pad is made of cellulose or cross-linked silica. A conjugate release pad is disposed in close contact with the strip material and the sample application pad. Antibody or analyte coated microparticles are deposited onto the conjugate release pad and dried down for stable long term use as outlined above.

As indicated above, in the Examples described below, a specific antibody labelled gold nanoparticle is dried on the pad and after addition of the sample, the labelled particle interacts with the fluid flow both mobilising the gold particles and enabling specific interactions that are initiated and continue during the chromatographic process. The liquid moves under the capillary force of the strip material and the absorbent pad attached at the distal side of the strip maintains liquid flow by wicking the liquid towards the end of the strip.

As exemplified, the particles used in the assay are colloidal gold but those skilled in the art will appreciate that other particles can be used such as latex, silver, liposomes, selenium or carbon can also be used. In addition where detection is based on purely visual detection, assays can be interpreted by reading colour intensity, and alternative labels can also be used such as dyes.

Where automated reading of test strips is used, .e.g. for quantitative LFA, the labels described above can also be used, with additional options for application of non-visual fluorescent or biochemiluminescent labels or other labels that include quantum dots and upconverting phosphor technology which offer other forms of particles.

As indicated above, in the present invention, more than one line is generally employed—at least one test line and an optional control line. At the test line, the combination of the sample analyte and the reporter results in the required response. A response at a control line confirms a proper flow of the liquid through the strip.

Materials suitable for use as conjugate pads include glass fibre filters, polyester, rayon, cellulose filters, and surface-treated (hydrophilic) polyester, polypropylene filters or other synthetic materials. Examples of such materials include Asymmetric Polysulphonone A supplied by PALL or Rapid 24/27 supplied by Whatman and conjugate pads available from MDI.

Materials suitable for use as blood separation pads or prefilters which generate high quality plasma include microporous materials that remove blood cells and deliver plasma to an IVD test strip or microfluidic channel without haemolysis or binding of diagnostic biomarkers.

In addition to the methods described in the Examples outlined below, other combinations are also possible in the assay of the invention. One such alternative competitive assay format requires a combination of an antibody labelled with a specific binding partner 1, such as biotin, and antigen coated gold deposited on a conjugate pad. In addition, one or more lines are printed on membranes with a complementary binding partner 2, for example streptavidin or similar, which will react with binding partner 1.

The second binding partner 2 can be printed as a single line or as multiple lines so that one or several lines can be generated in the test. In this format, where no analyte is present or is below a threshold, following mobilisation by addition of sample, the labelled antibody and analyte coated gold will react to form an antibody-analyte gold complex which migrates from the conjugate pad to the membrane so that the complex reaches the printed second binding partner where a reaction occurs between the first binding partner and the second generating a clear line. When carefully optimised, unbound antibody-gold complex migrates past the first test line. Where a second, or more test lines are printed with second binding partner, the antibody-gold complex will also react generating more than one test line.

However, if the sample added contains analyte, or where analyte is present above a defined threshold level, the analyte in the sample reacts with the labelled antibody, competing with analyte labelled gold, with competition increasing as the level of analyte in the sample increases. On reaching the test lines, the analyte-labelled antibody can also react with the second binding partner such that less antibody-gold complex can react, causing a reduction in the intensity of colour at the test line. As analyte in the sample increases further, the competition between analyte and analyte-gold for reaction with the labelled antibody increases further so that at a particular level of analyte, all the labelled antibody reacts with the analyte in the sample, with no labelled antibody available to react with the analyte-gold. As a result no test line or lines will be visible as the reaction at the test line.

It will be clear to those skilled in the art that the test can also use antibody gold particle and analyte labelled with binding partner 1. Further, other combinations of binding partner are also possible.

### Example 1

The presence of a hook effect in LFA's of the prior art and the elimination of the hook effect using an LFA in accordance with the invention employing SAA as an analyte was demonstrated as follows.

The hook effect was demonstrated in the analysis of SAA employing a sandwich assay using standard lateral flow technology test format well known to those skilled in the art. While variations in test assembly are known the example given is descriptive of typical analytical approaches adopted in the prior art.

Test strips were prepared as follows:

Antibody-gold nanoparticle conjugates were prepared using typical known methods as referenced in Conjugation of colloidal gold to proteins, Methods in Mol Biol, 2010, 588, 369-373. Briefly, 1 ml of gold nanoparticles (40 nm gold particles, BBI, Cardiff, UK) were coated with 100 μl monoclonal antibody to SAA at 0.5 mg/ml and incubated for 1 hour at room temperature. Unbound antibody was removed by centrifugation at 2500 rpm. The pellet washed twice in 20 mM borate buffer 4, pH 8 after 2×5 minute washes in 20 mm borate buffer, pH 8, and the final pellet was re-suspended in the same buffer containing 10% sucrose.

Membranes were also prepared using methods well known to those skilled in the art. Briefly, High Flow 135, 30 cm×2.5 cm (Millipore), backed with plastic card backing for support (30 cm×7.5 cm) were printed with monoclonal antibody to SAA at 0.5 mg/ml, 0.1 μl per test strip using an Isotron printing system, and allowed to air dry for 1 hour resulting in a single test line 30 cm long. Strips of adsorbent pads (Ahlstrom 222, 30 cm×2.2 cm) were placed on the plastic backed membrane so that there was contact between the membrane and the adsorbent material. Similarly, conjugate pad material (treated polyester, PT-R6, 30 cm×2 cm, MDI, India), was placed onto the plastic backed membrane so that conjugate pad and membrane were also in direct contact with the High Flow membrane. Finally sample prefilter (FR-1, 0.35, 30 cm×2 cm, MDI, India) was placed so that it was in contact with the conjugate pad. Hence the final plastic backed cards were provided with a sample pre-filter in contact with the conjugate pad, which in turn was in contact with the High Flow membrane, which in turn was in contact with the adsorbent material.

The cards were subsequently cut into 75 mm×4 mm test strips. Finally 2 μl of Monoclonal anti-SAA gold conjugate was deposited onto the conjugate pad of each test strip, air dried before running the test strip. The test strips were inserted in plastic cassettes to facilitate test evaluation as indicated below. The cassettes used are well known to those skilled in the art and typically have a sample port or window at which sample and optionally running buffer is added with test results appearing in a test window which is downstream of the sample port.

Samples containing SAA were prepared in PBS to give a range of concentrations of 0 ng/ml, 10 ng/ml, 100 ng/ml, 1000 ng/ml, 10,000 ng/ml, 100,000 ng/ml and 1,000,000 ng/ml. 5 μl of sample was applied to the end of the test strip followed by addition of 100 μl of a PBS buffer. Results were observed at 10 minutes. In the absence of a hook effect lines were expected to appear as the concentration of SAA in samples increased.

As expected no signal was seen at 0 ng/ml or 10 ng/ml due to sensitivity limitations. Signal was seen as the concentration of SAA increased between 100 ng/ml and 10,000 ng/ml. However, at higher concentrations such as 100,000 ng/ml or 1,000,000 ng/ml no signal was observed clearly indicating the presence of a hook effect as SAA concentration increased to levels that would be expected in clinically relevant samples from animals or humans with an inflammatory condition.

Test strips were also run with equine serum samples which had been shown to contain SAA at <5 μg/ml, 22 μg/ml, 500 μg/ml and 1250 μg/ml using a laboratory based assay system (SAA TIA; LZ-SAA, Eiken Chemical Co., Tokyo, Japan). Analyses were performed on an automated analyser (ADVIA 1650 Chemistry System, Bayer, Newbury, UK) according to the manufacturer's recommendations. Calibration curves were created using a human SAA calibrator from the same manufacturer (Eiken Chemical Co.). 5 μl of serum was applied to a test strip followed by 100 μl of a PBS buffer. Results were read after 10 minutes. No signal was seen at 0 μg/ml, 500 ug/ml, or 1250 μg/ml but was seen at 22 μg/ml indicating a clear hook effect had occurred.

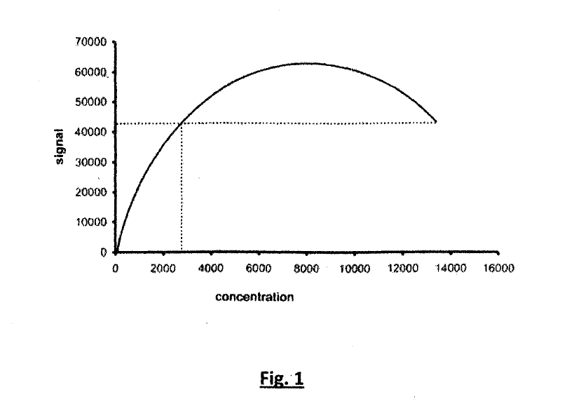

FIG. 1 shows a diagram illustrative of the hook effect described above in which analyte concentration is plotted on the X-axis and analyte signal is plotted on the Y-axis with the signal decreasing at high concentrations.

The above analysis was repeated employing an LFA in accordance with the invention as follows. The samples used to demonstrate the presence of a hook effect were subsequently run on test strips in accordance with the invention as outlined below.

While other variations in test assembly are possible, such as using combined sample/conjugate pad or a single material for conjugate and test line deposition, the following description is indicative of one approach.

Gold particles were conjugated using methods well known to those skilled in the art (e.g. Oliver C, Conjugation of colloidal gold to proteins, Methods in Mol Biol, 2010, 588, 369-373). Briefly, 40 nm gold particles (BBI, Cardiff, UK) were coated with a monoclonal antibody to SAA at 0.1 mg/ml in 20 mm borate buffer, pH 8 as indicated above. In addition a second gold particle (40 nM, BBI, Cardiff, UK) was coated with mouse anti-chicken IgY monoclonal antibody at 0.1 mg/ml. This second gold particle was used to generate a control line to enable visual observation of the control line.

As shown in FIG. 3, test strips, 75 mm×4 mm in dimension, were made according to well established methods and test formats. The test strip was composed of a sample prefilter 5 (FR-1, 0.35, MDI, India) to remove blood cells, directly in contact with a conjugate pad 6 (Treated polyester, PTR7, MDI, India) onto which 2 μl of anti-SAA monoclonal gold conjugates and 0.25 μl of anti-IgY gold conjugate was applied. The conjugate pad in turn was in direct contact with a membrane material 7 (SS-12 Nitrocellulose, MDI, India), on which Test Lines 1,2,3 were printed, and finally an adsorbent material 8 (Ahlstrom 222, 30 cm×2.2 cm) which was directly in contact with the membrane. SAA was printed onto the test strip using standard spraying methodology using either an Isotron printing system.

In this example, SAA was printed as three Test Lines 1,2,3. The Test Lines 1,2,3 were printed so that Test Line 1 (T1) was closest to the end of the test strip at which sample was added, Test Line 2 (T2) was downstream of Test Line 1 and Test Line 3 (T3) was downstream of Test Line 2. A Control Line 4 was located downstream of Test Line 3 consisting of purified chicken IgY antibody printed at 0.25 mg/ml although several other methods for generation of control lines will be known to those skilled in the art.

The concentration of SAA printed onto each test strip increased from Test Line 1 to Test Line 2 to Test Line 3. The concentration of SAA at Test Line 1 was 5 μg/ml, at Test Line 2 30 μg/ml and at Test Line 3 300 μg/ml. (However, the concentration of SAA at the test lines is not restricted to those used in this example).

Finally, test strips were inserted in plastic cassettes to facilitate test evaluation as indicated below. The cassettes used had a sample port or window at which sample and optionally running buffer was added with test results appearing in a test window downstream of the sample port.

The test was designed such that in the absence of analyte, or when the analyte was present at low levels or below a threshold, the four Test and Control Lines 1,2,3,4 appeared in the test window where the intensity of colour on T1 was less than T2 which in turn was less than (or equal to) T3 while the Control Line 4 always appeared if a test was run correctly.

5 μl of sample was applied directly onto the test strip via the sample application port on the cassette, followed by 100 μl of PBS buffer. After 10 minutes results were observed and interpreted. Samples with SAA at 0, 10 ng/ml, 100 ng/ml and 1000 ng/ml resulted in three clearly visible Test Lines 1,2,3 with increasing colour intensity from T1-T3. However as the concentration of SAA increased in the samples to 10,000 ng/ml and higher, the intensity of colour on the Test Lines 1,2,3 decreased sequentially such that at certain concentrations of SAA, T1 was no longer visible so that only two Test Lines 2,3 were visible indicating higher SAA in a sample. Likewise, as the sample concentration increased further to 100,000 ng/ml, the intensity of the remaining Test Lines decreased such that at certain concentration only one Test Line, T3, was visible. As the concentration of SAA increased further to 1,000,000 ng/ml, no test lines were visible indicating the level of analyte in a sample was very high. Accordingly, no false low or false negative results were observed with either spiked SAA in buffer or with serum samples with known levels of SAA thus indicating that the LFA of the invention overcame the hook effect.

Accordingly, it has now surprisingly been demonstrated that an LFA in accordance with the invention can be performed on APP's such as SAA having multiple epitopes employing a simple competitive assay format to obtain qualitative and semi-quantitative results that do not suffer from the hook effect without requiring complex processing steps such as washing or dilution. Due to the simplicity of the LFA of the invention, LFA devices can be used in-situ to obtain rapid and immediate results without requiring the use of laboratory equipment or personnel.

### Example 2

The presence of a hook effect in lateral flow tests using whole blood samples with SAA as an analyte was demonstrated as follows.

Membranes with three test lines were prepared as indicated in Example 1. Samples with SAA at <5 μg/ml, 39 μg/ml, 188 μg/ml and greater than 500 μg/ml as determined by the laboratory method described in Example 1 were investigated. The test was run using 10 μl of sample added to test strips followed by 100 μl of PBS, pH 7.2. Tests were read visually after 10 minutes. No signal was seen at samples less than 5 μg/ml SAA or with SAA samples at 188 μg/ml or greater 500 μg/ml of SAA although a signal was observed when using the sample at 39 μg/ml clearly indicating the presence of a hook effect with whole blood samples.

The analysis of the samples was repeated with LFA test strips in accordance with the invention as described in Example 1. 10 μl of sample was added to the sample port followed by 100 μl of PBS buffer. Three test lines were observed with the sample<5 μg/ml, two test lines were observed with the sample containing 39 μg/ml SAA, one test line was observed using the sample at 188 μg/ml and no test lines were observed with a sample at >500 μg/ml, clearly demonstrating that the LFA of the invention overcame the hook effect experienced with whole blood.

### Example 3

The use of the assay of the invention in the analysis of SAA in equine blood samples to determine the inflammatory status of the horse for diagnostic purposes was demonstrated as follows.

For rapid test analysis, whole blood analyses were performed with test strips with three Test Lines 1,2,3 as described in Example 1. 10 μl of whole blood was applied directly onto the test strip via the sample application port on the cassette followed by 100 μl of PBS buffer. After 10-15 minutes results were observed and interpreted as “Normal” (three Test Lines 1,2,3 and Control Line 4 visible), “Mild Inflammation” (two Test Lines 2,3 and Control Line 4 visible), “Moderate Inflammation” (one Test Line 3 and Control Line 4 visible) and “Severe Inflammation” (Control Line 4 only visible).

Corresponding serum samples from each blood sample were also analyzed using a commercially available a laboratory based system to establish SAA levels. SAA concentrations were determined using a human turbidimetric immunoassay (SAA TIA; LZ-SAA, Eiken Chemical Co., Tokyo, Japan) and analyses were performed on an automated analyser (ADVIA 1650 Chemistry System, Bayer, Newbury, UK) according to the manufacturer's recommendations. Calibration curves were created using a human SAA calibrator from the same manufacturer (Eiken Chemical Co.). Visual results observed in the rapid assay device correlated with the quantitative results obtained with the commercial assay.

The results showed that the rapid LFA of the invention identified those samples that were from normal healthy horses and distinguished them from horses that had an active inflammatory condition based on laboratory analysis of SAA without being compromised by a hook effect.

TABLE 1
Analysis of Equine SAA Levels

| | SAA/(Laboratory assay) | |
|---|---|---|
| Sample | (μg/ml) | SAA/(LFA of the invention) |
| 1 | <5 | Normal |
| 2 | >500 | Severe |
| 3 | <5 | Normal |
| 5 | <5 | Normal |
| 6 | 5.04 | Normal |
| 7 | <5 | Normal |
| 8 | 188.5 | Moderate |
| 9 | 32.9 | Mild |
| 10 | 348.8 | Severe |
| 11 | <5 | Normal |
| 12 | <5 | Normal |
| 13 | 196.5 | Moderate |
| 14 | 7.6 | Normal |
| 15 | 5.8 | Normal |
| 16 | 173 | Moderate |
| 17 | 198 | Moderate |
| 18 | 7.7 | Normal |
| 19 | <5 | Normal |
| 20 | >500 | Severe |
| 21 | 324 | Severe |
| 22 | <5 | Normal |
| 23 | >500 | Severe |
| 24 | >500 | Severe |
| 25 | >500 | Severe |
| 26 | <5 | Normal |
| 27 | 341.8 | Severe |
| 28 | 348 | Severe |
| 29 | 87.9 | Mild |

### Example 4

The use and efficacy of the LFA of the invention in rapid animal side diagnoses was demonstrated as follows.

Rapid tests were prepared as described in Example 1 using three Test Lines 1,2,3 and a Control Line 4 as previously described. Blood samples were collected from six horses undergoing surgery. The blood was collected and tested animal side to assess inflammatory status. The blood was collected into standard serum collection tubes. As blood was analysed immediately it was not necessary to use any particular type of specialised blood collection tube. However, where required, collection of blood in tubes containing anticoagulants such as EDTA or heparin is equally possible without affecting the outcome of the result.

5 μl of whole blood was applied to the test trip using a plastic disposable micropipette (Microsafe tubes, Safe-Tec, USA). Results were read within 15 minutes. Samples were also subsequently analysed using a laboratory based assay as described in Example 1.

TABLE 2
Animal side analysis of Equine SAA Levels

| | SAA/(Laboratory assay) | |
|---|---|---|
| Sample | (μg/ml) | SAA/(LFA of the invention) |
| 1 | <5 | Normal |
| 2 | <5 | Normal |
| 3 | <5 | Normal |
| 4 | >500 | Severe |
| 5 | 348 | Severe |
| 6 | 80 | Mild |

### Example 5

The use of the LFA of the invention in the analysis of SAA in equine synovial fluid was demonstrated as follows.

Synovial fluid samples were collected from 19 horses and samples were run directly on the rapid LFA tests prepared as described in Example 1 using three Test Lines 1,2,3 and a Control Line 4. Twelve samples were taken from normal healthy joints of horses and were not expected to have any active inflammatory condition. Seven samples were taken from joints of horses under investigation for lameness of unknown origin. In six of the seven samples no active inflammation was detected and results were supported by subsequent laboratory analysis for SAA. In a seventh sample, the rapid assay of the invention indicated a severe inflammatory condition and laboratory analysis confirmed that SAA was greater than 500 μg/ml. Clinically, the horse was shown to have a peri-articular abscess leading to inflammation in the joint which was also confirmed by cytology analysis performed by a reference laboratory.

### Example 6

Use of the LFA of the invention to assess inflammatory conditions in cats by reference to SAA levels was demonstrated as follows.

Blood samples were taken from 17 cats undergoing routine investigation. Samples were analyzed both using the assay of the invention as described in Example 1 using three Test Lines 1,2,3 and a Control Line 4. In addition, samples were also analyzed by a commercially available laboratory based test for SAA (Eiken, Japan) as described in Example 1. The assay results were categorized as normal, mild, moderate or severe inflammation.

TABLE 3
Analysis of Feline SAA Levels

| | SAA/(laboratory assay) | SAA/(LFA of the |
|---|---|---|
| Sample | (μg/ml) | invention) |
| 1 | 0.3 | Normal |
| 2 | 37.1 | Mild |
| 3 | 71 | Mild |
| 4 | 120 | Moderate |
| 5 | 0.6 | Normal |
| 6 | 185 | Moderate |
| 7 | 0.6 | Normal |
| 8 | 212 | Severe |
| 9 | 0 | Normal |
| 10 | 0.8 | Normal |
| 11 | 178 | Moderate |
| 12 | 103 | Moderate |
| 13 | 1 | Normal |
| 14 | 0.7 | Normal |
| 15 | 0.3 | Normal |
| 16 | 0.1 | Normal |
| 17 | 48.6 | Mild |

### Example 7

The use of the assay of the invention to assess inflammatory conditions in humans using SAA was demonstrated as follows.

Samples were taken from eight humans, five with no indication of any health condition. Assays of the invention using three Test Lines 1,2,3 were prepared as indicated in Example 1. Blood samples were taken using a blood lancet and applied directly to the LFA using a disposable sample applicator (Microsafe tubes, Safe-Tec, USA). Additional sample was collected into microtubes for laboratory analysis of SAA levels. The five samples from the healthy individuals gave a normal SAA response in the rapid test. These were also shown to have low levels of SAA based on laboratory analysis. The sample from the 6^{th} person with fever, high temperature and abdominal pain gave a severe inflammatory condition on the rapid test, with laboratory analysis demonstrating SAA above the level of the reference range. Two additional samples were collected from two people with signs of colds, high temperature and feelings of ill health. Blood samples were taken using a blood lancet and applied directly to the rapid tests with both samples showing severe inflammation. Additional sample was collected into microtubes for laboratory analysis to assess SAA levels. Both samples were shown to have SAA above the reference range of the assay. The following day both individuals were diagnosed with infections and prescribed antibiotics after consultation with a medical practitioner.

### Example 8

The use of the LFA of the invention to assess inflammatory conditions in cows using SAA was demonstrated as follows.

Test strips were prepared as described in Example 1 using three Test Lines 1,2,3 and a Control Line 4. Ten blood samples were taken from cows and tested for inflammatory status using a laboratory based ELISA for detection of SAA. The samples were also assessed for inflammatory status using the assay of the invention by application of 5 μl to the test strip followed by 2 drops of buffer. All tests were read at 15 minutes.

TABLE 4
Analysis of Bovine SAA Levels

| Sample | SAA level (μg/ml) | SAA/(LFA of the invention) |
|---|---|---|
| 1 | 110 | Severe |
| 2 | 0 | Normal |
| 3 | 14 | Normal |
| 4 | >150 | Severe |
| 5 | 0 | Normal |
| 6 | 12.5 | Normal |
| 7 | 0 | Normal |
| 8 | 0 | Normal |
| 9 | 18 | Normal |
| 10 | 125 | Severe |

In short, the LFA and device of the present invention enjoys a number of advantages over the prior art. Firstly, the LFA is adapted for use with whole blood as well as other bodily fluids such as serum, plasma, colostrums and milk. Secondly, a competitive assay format is employed so that, as analyte concentration increases, signal generally decreases so that increasing target analyte levels in a sample results in a gradual reduction in signal (in contrast to prior art assays which employ a direct relationship between signal and analyte concentration typically in a non-competitive sandwich assay format subject to the hook effect). Thirdly, signal generation is based on the use of multiple test lines (typically 2 to 4), with the option of an additional control line to facilitate semi-quantitative analyses.

Typically, three test lines are used for SAA analysis purposes so that three visible signal lines is indicative of a normal healthy horse, no visible test lines is indicative of severe inflammation while intermediate combinations are indicative of a problem that may require further monitoring or intervention. The distinctions can be categorized by reference ranges for each. Fourthly, assay results are complete in about 10-15 minutes with normal healthy animals giving a result in less than about 3 minutes.

Accordingly, the immediate availability of test results in-situ or animal side within 10 minutes and up to within 2-3 minutes facilitates a meaningful semi-quantitative diagnostic and prognostic test to assist in an almost immediate or real-time determination of an animal's (or human's) health status.

The invention is not limited to the embodiments herein described which may be varied in construction and detail without departing from the scope of the invention.

1. A method for eliminating the hook effect in the detection of a target analyte in a sample in which the target analyte comprises a member of a specific binding pair comprising:
applying the sample to a solid phase carrier material, the solid phase carrier material having labelled first or second members of the specific binding pair thereon and complimentary immobilised first or second members of the specific binding pair downstream of the labelled first or second members of the specific binding pair,
generating a signal at the complimentary immobilised first or second members of the specific binding pair in accordance with downstream movement of the labelled first or second members and the target analyte to bind with the complimentary immobilised first or second members, and
detecting the presence of the target analyte in accordance with the signal generated at the complimentary immobilised first or second members.

2. (canceled)

3. (canceled)

4. A method as claimed in claim 1 wherein the analyte is an animal analyte.

5. A method as claimed in claim 4 wherein the animal analyte is sampled from the group comprising humans, horses, cows, dogs, cats, pigs, cattle, goats, sheep, donkeys, and llamas.

6. A method as claimed in claim 5 wherein the animal analyte comprises a protein.

7. A method as claimed in claim 6 wherein the protein comprises an acute phase protein.

8. A method as claimed in claim 7 wherein the acute phase protein comprises serum amyloid A.

9. (canceled)

10. A method as claimed in claim 2 wherein the sample comprises a bodily fluid.

11. A method as claimed in claim 10 wherein the method further comprises the step of pre-filtering the bodily fluid.

12. (canceled)

13. (canceled)

14. A method as claimed in claim 11 wherein the bodily fluid comprises whole blood.

15. A method as claimed in claim 2 wherein the signal is generated at at least one test line.

16. (canceled)

17. A method as claimed in claim 15 wherein the analyte is quantitatively or semi-quantitatively detected in accordance with the signal generated at the test line.

18. (canceled)

19. A method as claimed in claim 1 wherein the analyte comprises an immunologically detectable analyte, the immunogically detectable analyte being the first member of the specific binding pair and the second member of the specific binding pair comprising immobilised antibody.

20-25. (canceled)

26. A method as claimed in claim 1 further comprising the step of diagnosing a condition in a human or animal in accordance with the signal generated.

27.-30. (canceled)

31. A lateral flow assay device for eliminating the hook effect in the detection of a target analyte in a sample in which the target analyte comprises a member of a specific binding pair comprising:
a solid phase carrier material;
labelled first or second members of the specific binding pair on the solid phase carrier material;
complimentary immobilised first or second members of the specific binding pair on the solid phase material downstream of the labelled first or second members of the specific binding pair;
a signal being generatable at the complimentary immobilised first or second members of the specific binding pair in accordance with downstream movement of the labelled first or second members to bind with the complimentary immobilised first or second members, and
a pre-filter on the solid phase carrier material to remove interferences from the sample.

32. A lateral flow assay device as claimed in claim 31 wherein the labelled first or second members of the specific binding pair on the solid phase carrier material comprises a labelled target analyte antigen and the complimentary immobilised first or second members of the specific binding pair on the solid phase material downstream of the labelled first or second members of the specific binding pair comprises an antibody to the antigen.

33. A lateral flow assay device as claimed in claim 32 wherein the antigen comprises a protein.

34. A lateral flow assay device as claimed in claim 33 wherein the protein comprises an acute phase protein.

35. (canceled)

36. A lateral flow assay device as claimed in claim 34 wherein the acute phase protein comprises an animal acute phase protein.

37. A lateral flow assay device as claimed in claim 36 wherein the animal acute phase protein is selected from the group comprising human, equine, bovine, canine, feline, porcine, goat, sheep, donkey and llama acute phase protein.

38. A lateral flow assay device as claimed in claim 37 wherein the acute phase protein comprises serum amyloid A.

39-41. (canceled)
